(19) United States
(12) Patent Application Publication
(10) Pub. No.: US 2026/0144501 A1
(43) Pub. Date: May 28, 2026

(54) MEDICAL IMAGING SYSTEM WITH A RAIL SYSTEM

(71) Applicant: Siemens Healthineers AG, Forchheim (DE)

(72) Inventors: Elmar GARCIA, Nuernberg (DE); Markus KOERBER, Buttenheim (DE); Jens FEHRE, Hausen (DE)

(73) Assignee: Siemens Healthineers AG, Forchheim (DE)

(21) Appl. No.: 19/400,145

(22) Filed: Nov. 25, 2025

(30) Foreign Application Priority Data
Nov. 27, 2024 (DE) 10 2024 211 348.4

(51) Int. Cl. A61B 6/03; A61B 6/00

(52) U.S. Cl. CPC A61B 6/035; A61B 6/4435

(57) ABSTRACT

One or more example embodiments relates to a medical imaging system comprising a computed tomography gantry; a carriage; a rail system; and a support profile, wherein the computed tomography gantry is moveable via the carriage and the rail system such that a translation movement of the computed tomography gantry relative to a base surface occurs along the rail system, the support profile is form-fit into a depression that is relative to the base surface, the support profile has a rail groove to form-fit receive a rail of the rail system, and the rail of the rail system is form-fit into the rail groove such that the rail of the rail system does not protrude above the base surface in relation to a vertical direction.

### CROSS-REFERENCE TO RELATED APPLICATION(S)

The present application claims priority under 35 U.S.C. § 119 to German Patent Application No. 10 2024 211 348.4, filed Nov. 27, 2024, the entire contents of which is incorporated herein by reference.

### Field

One or more example embodiments relates to a medical imaging system.

### Related Art

Rails of rail-guided medical devices are often installed in the floor with covers or are installed on the floor. The former require coverings against liquids, dust or suchlike. The latter can be perceived as unpleasant due to the elevation relative to the floor level (in particular with elevations of up to 3 mm) or can act as trip hazards (in particular with elevations of greater than 3 mm).

As prior art, reference is made to DE 10 2023 202 908 A1.

### SUMMARY

One or more example embodiments enables a movement of a computed tomography gantry that is improved with regard to the installation, maintenance and/or cleaning capability of the components involved.

Independent of the grammatical term usage, individuals with male, female or other gender identities are included within the term.

### BRIEF DESCRIPTION OF THE DRAWINGS

Example embodiments will now be described, making reference to the accompanying drawings. The illustrations in the figures are schematic, greatly simplified and not necessarily to scale.

FIG. 1 shows a support profile, a rail and a measuring track in a first view according to one or more example embodiments.

FIG. 2 shows the support profile, the rail and the measuring track in a second view according to one or more example embodiments.

FIG. 3 shows a medical imaging system with a computed tomography gantry, a carriage and a rail system according to one or more example embodiments.

FIG. 4 shows a support profile and a rail with an anti-rotation device according to one or more example embodiments.

FIG. 5 shows a flow diagram of a method for moving a computed tomography gantry according to one or more example embodiments.

### DETAILED DESCRIPTION

One or more example embodiments relates to a medical imaging system having a computed tomography gantry, a carriage, a rail system and a support profile,
wherein the computed tomography gantry is movably mounted via the carriage and the rail system such that a translation movement of the computed tomography gantry relative to a base surface can be carried out along the rail system,
wherein the support profile is received in a form-fitting manner into a depression that is formed relative to the base surface,
wherein the support profile has a rail groove for form-fitting receiving of a rail of the rail system,
wherein the rail of the rail system is received in a form-fitting manner into the rail groove such that the rail of the rail system does not protrude above the base surface in relation to a vertical direction.

The rail of the rail system is thus not disturbing when stepped on and does not need a covering against liquids and/or dust which often require servicing due to the mechanical loadings. Furthermore, the open design of the rails is more readily accessible for cleaning operations.

Optionally, it can be provided that no tangent plane to the rail of the rail system that is parallel to the base surface extends above the base surface.

Optionally it can be provided that the base surface has a first base surface region and a second base surface region,
wherein the first base surface region and the second base surface region are coplanar with one another,
wherein the depression is situated between the first base surface region and the second base surface region,
wherein the rail of the rail system is received in a form-fitting manner into the rail groove such that the rail of the rail system does not protrude above the first base surface region and that the rail of the rail system does not protrude above the second base surface region.

In particular, it can be provided that the depression is situated, in relation to a transverse direction that is perpendicular to a longitudinal direction of the rail of the rail system and/or parallel to the base surface, between the first base surface region and the second base surface region. For example, the first base surface region and the second base surface region are separated from one another by the depression.

In particular, it can be provided that the tangent plane parallel to the base surface does not extend, at the highest point of the rail of the rail system, above the first base surface region and/or above the second base surface region. In particular, it can be provided that the tangent plane parallel to the base surface is coplanar, at the highest point of the rail of the rail system, with the first base surface region and/or the second base surface region.

Optionally, it can be provided that the support profile has a connecting area, wherein the support profile is received in a form-fitting manner into the depression such that the connecting area is joined steplessly, in particular, in coplanar manner, to the base surface, in particular, as a continuation of the base surface.

The continuation of the base surface can, in particular, be continuous. In particular, it can be provided that the tangent plane parallel to the base surface at the highest point of the rail of the rail system is coplanar with the base surface and/or with the connecting area.

Optionally it can be provided that the connecting area has a first connecting area region and a second connecting area region,
wherein the rail of the rail system is situated between the first connecting area region and the second connecting area region,
wherein the support profile is received in a form-fitting manner into the depression such that the first connecting area region is joined steplessly, in particular, in coplanar manner, to the first base surface region, in particular, as a continuation of the first base surface region and that the second connecting area region is joined steplessly, in particular, in coplanar manner, to the second base surface region, in particular, as a continuation of the second base surface region.

The continuation of the first base surface region can, in particular, be continuous. The continuation of the second base surface region can, in particular, be continuous. In particular, it can be provided that the rail of the rail system is situated, in relation to a transverse direction that is perpendicular to a longitudinal direction of the rail of the rail system and/or is parallel to the base surface between the first connecting area region and the second connecting area region. The first connecting area region and the second connecting area region can be, for example, coplanar with one another.

Optionally, it can be provided that the base surface extends substantially horizontally,
wherein the rail of the rail system is received in a form-fitting manner into the rail groove such that the rail of the rail system does not protrude above the base surface in relation to the vertical direction.

In particular, it can be provided that the tangent plane parallel to the base surface at the rail of the rail system is horizontal and/or that the connecting area extends substantially horizontally. In particular, it can be provided that the longitudinal direction of the rail of the rail system is horizontal and/or that the transverse direction that is perpendicular to the longitudinal direction of the rail of the rail system and/or is parallel to the base surface, is horizontal. In particular, it can be provided that the highest point of the rail of the rail system in relation to the vertical direction is arranged not higher than the base surface.

Optionally, it can be provided that the rail of the rail system is a first rail of the rail system and is arranged parallel to a second rail of the rail system,
wherein the base surface is substantially parallel to a rail plane, wherein the rail plane extends through the first rail of the rail system and through the second rail of the rail system.

Optionally, it can be provided that, in a transverse plane that is perpendicular to a longitudinal direction of the rail of the rail system, the rail of the rail system has a round profile, in particular, a circular profile.

In particular, it can be provided that, in a transverse plane that is perpendicular to a longitudinal direction of the rail of the rail system, the rail of the rail system has a convex profile, in particular, a circular profile.

Optionally, it can be provided that, in a transverse plane that is perpendicular to a longitudinal direction of the rail of the rail system, the rail of the rail system has a rounded rectangular profile.

Optionally, it can be provided that, in the region of the rail groove, the support profile has a profile-side connecting element,
wherein the rail of the rail system has a rail-side connecting element that is configured corresponding to the profile-side connecting element,
wherein the rail of the rail system is secured by way of a form-fitting of the profile-side connecting element and of the rail-side connecting element against a rotation about a longitudinal axis of the rail of the rail system relative to the support profile.

One or more example embodiments further relates to a medical imaging system having a computed tomography gantry, a carriage, a rail system and a support profile,
wherein the computed tomography gantry is mounted able to be moved via the carriage and the rail system such that a translation movement of the computed tomography gantry relative to a base surface can be carried out along the rail system,
wherein the support profile is received in a form-fitting manner into a depression that is formed relative to the base surface,
wherein the support profile has a rail groove for form-fitting receiving of a rail of the rail system,
wherein, in the region of the rail groove, the support profile has a profile-side connecting element,
wherein the rail of the rail system has a rail-side connecting element that is configured corresponding to the profile-side connecting element,
wherein the rail of the rail system is secured by way of a form-fitting of the profile-side connecting element and of the rail-side connecting element against a rotation about a longitudinal axis of the rail of the rail system relative to the support profile.

Optionally, it can be provided that the profile-side connecting element protrudes in a rod-like manner toward the rail of the rail system in a direction that is perpendicular to the longitudinal axis of the rail of the rail system,
wherein the rail-side connecting element has a cut-out for receiving the profile-side connecting element.

Optionally, it can be provided that the rail-side connecting element protrudes in a rod-like manner away from the rail of the rail system in a direction that is perpendicular to the longitudinal axis of the rail of the rail system,
wherein the profile-side connecting element has a cut-out for receiving the rail-side connecting element.

Optionally, it can be provided that the cut-out is a bore, in particular, in the form of a circular hole.

Optionally, it can be provided that the cut-out is a slot and/or extends longitudinally parallel to the longitudinal axis of the rail of the rail system.

In particular, it can be provided that the rail of the rail system can be received into the rail groove in a form-fitting manner in that the rail of the rail system is laid into the rail groove, in particular, by way of a lowering the rail of the rail system relative to the support profile in a vertical lowering direction perpendicular to the longitudinal axis of the rail of the rail system. For this purpose, the support profile can be constructed, for example, free of undercuts and/or can be constructed in multiple parts such that undercuts only form after the insertion of the rail of the rail system into the rail groove and the subsequent joining of the support profile.

Optionally, it is provided that the carriage and the rail system are constructed to transmit a drive force for the translation movement of the computed tomography gantry in a force-locked manner from the carriage to the rail system, in particular, by way of friction.

In particular, it can be provided that the rail system is static relative to a base surface and/or is firmly anchored relative to the base surface. In particular, it can be provided that an examination object is static relative to the rail system and/or relative to the base surface. The rail system can form, in particular, a linear guide for the carriage.

For example, the medical imaging system can have an examination table for supporting the examination object. The examination table can be static, in particular, relative to the rail system and/or relative to the base surface and/or can be firmly anchored relative to the rail system and/or relative to the base surface. The examination object can be, for example, a person to be investigated, in particular, a patient and/or can be supported on the examination table, in particular, mounted static relative to the examination table.

The translation movement can take place, in particular, relative to the rail system, relative to the base surface, relative to the examination table and/or relative to the examination object. The translation movement can be, in particular, substantially horizontal. The base surface can be, in particular, substantially horizontal. The base surface can be, in particular, a floor of an examination room and/or made of concrete.

The computed tomography gantry can have, for example, a support frame and a rotor that is mounted able to be rotated relative to the support frame, wherein the radiation source and the radiation detector are arranged on the rotor. Optionally, the computed tomography gantry can have a tilt frame that is mounted able to be tilted relative to the support frame, wherein the rotor is arranged on the tilt frame. The radiation source and the radiation detector can cooperate for a recording of a projection dataset of the examination object. The computed tomography gantry can have, for example, an opening. In particular, the rail system, the examination table and the opening can be arranged relative to one another such that by way of the translation movement of the computed tomography gantry, the examination table is introduced into the opening, in particular, together with the examination object supported on the examination table.

One embodiment provides that a set of wheel-rail rolling contacts is formed between the carriage and the rail system, wherein the carriage and the rail system are constructed to transmit the drive force for the translation movement of the computed tomography gantry via the set of wheel-rail rolling contacts in a force-locked manner from the carriage to the rail system.

One embodiment provides that the set of wheel-rail rolling contacts receives the total weight force of the carriage and the computed tomography gantry, wherein each wheel-rail rolling contact that is included in the set of wheel-rail rolling contacts and receives at least a part of the total weight force of the carriage and of the computed tomography gantry transfers at least a part of the drive force for the translation movement of the computed tomography gantry in a force-locked manner, in particular, by way of friction.

In particular, it can be provided that the at least one part of the total weight force of the carriage and of the computed tomography gantry is not insignificant, for example, is greater than a tenth of the total weight force of the carriage and the computed tomography gantry. In particular, it can be provided that the at least one part of the drive force for the translation movement of the computed tomography gantry is not insignificant, for example, is greater than a tenth of the drive force for the translation movement of the computed tomography gantry.

In particular, the existence of a wheel-rail rolling contact can be precluded for the medical imaging system, although it receives a part of the total weight force of the carriage and the computed tomography gantry, transfers no part of the drive force for the translation movement of the computed tomography gantry.

The frictional force available for the drive depends upon the coefficient of friction and the normal force with which the friction wheel is loaded. In particular, if the wheel is driven directly and the friction of the wheel-rail rolling contacts is utilized, the total weight force of the carriage and of the computed tomography gantry can be utilized as the normal force. In the case of a friction wheel, a division of the weight force between the rail wheels and the friction wheel takes place so that only a part of the weight force is available as the normal force.

One embodiment provides that the rail system has a set of rails wherein the carriage has a set of wheels, wherein the set of wheels is arranged rolling on the set of rails. The set of rails can have, for example, the rail of the rail system. The set of rails can have, for example, the first rail of the rail system and/or the second rail of the rail system.

In particular, it can be provided that the set of rails and the set of wheels form the set of wheel-rail rolling contacts. In particular, it can be provided that each rail of the set of rails is a round rail and/or that each wheel of the set of wheels is a concave roller and/or is formed for rolling on a round rail. The rails and/or the wheels can be made, for example, from steel.

The round rails can be integrated into the floor, in particular, without a covering and without drive elements and thereby enable being driven over with patient beds and instrument trolleys. The drive force for the translation movement of the computed tomography gantry can be transferred, for example, on the basis of a force-locking, in particular, a frictional-locking between the wheels of the set of wheels and the rails of the set of rails of the carriage on the rail system.

One embodiment provides that, for each wheel of the set of wheels, the carriage has a wheel direct drive that cooperates with this wheel and contributes proportionally to the drive force for the translation movement of the computed tomography gantry.

In particular, it can be provided that, for each wheel of the set of wheels, the wheel direct drive that cooperates with this wheel directly drives this wheel and thereby contributes proportionally to the drive force for the translation movement of the computed tomography gantry. In particular, it can be provided that the drive force for the translation movement of the computed tomography gantry is generated by the wheel direct drives of the wheels of the set of wheels together. The wheel direct drive can have, for example, an electric motor, in particular, an electric wheel hub motor.

One embodiment provides that the medical imaging system also has a position measuring system and that the position measuring system is configured for generating a position information item, wherein the position information item relates to a position of the computed tomography gantry along the rail system.

The position of the computed tomography gantry along the rail system can be defined, in particular, relative to a reference point that is static relative to the base surface and/or relative to the rail system, in particular, during the translation movement of the computed tomography gantry relative to the base surface and/or relative to the rail system. In particular, it can be provided that the position of the computed tomography gantry along the rail system during the translation movement of the computed tomography gantry is measured continuously, in particular, with a sufficiently high sampling rate, in particular, such that the position information item for each projection dataset that has been recorded via the computed tomography gantry from an examination object during the translation movement of the computed tomography gantry, comprises a position of the computed tomography gantry at which this projection dataset has been recorded.

One embodiment provides that the position measuring system is configured for generating the position information item on the basis of a measurement, in particular, on the basis of a contactless measurement of the position of the computed tomography gantry along the rail system.

The contactless measurement can take place, for example, optically, magnetically, magnetostrictively, inductively and/or capacitively and/or can be based upon a runtime measurement. The combination of force-locked drive force transfer between, firstly, the wheel and the rail and, secondly, the contactless position measurement enables an exact positioning and position recognition of the carriage given a minimal engaging area, wherein both during drive and also during the position measurement, a form-fitting can be dispensed with. Thereby, the cleaning capability is improved and wear and tear reduced.

The measurement of the position of the computed tomography gantry along the rail system can take place, in particular, absolutely, for example, via an absolute value encoder, or incrementally, for example, via an incremental encoder.

One embodiment provides that the position measurement system has a measuring track and a position sensor, wherein the measuring track is static relative to the rail system and extends along the rail system, wherein the position sensor is connected to the carriage in such a way that it follows the translation movement of the computed tomography gantry and during the translation movement of the computed tomography gantry, cooperates with the measuring track, in particular, in order to carry out the measurement, in particular, the contactless measurement of the position of the computed tomography gantry along the rail system.

The measuring track can be, for example, a code track. The position sensor can be configured, for example, for sampling the code track. The measuring track can be, for example, a magnetic band. The magnetic band can be magnetized, in particular, at regular spacings. The measuring track can be, for example, a measuring band, in particular, a stainless steel measuring band and/or can be firmly anchored relative to the base surface.

One embodiment provides that the medical imaging system further has a support profile and that the support profile has a measuring track groove for receiving, in particular, for form-fitting receiving of the measuring track and the measuring track is received into the measuring track groove, in particular, in a form-fitting manner, wherein the support profile has a rail groove for form-fitting receiving of a rail of the rail system and the rail of the rail system is received into the rail groove in a form-fitting manner.

In particular, it can be provided that the support profile extends along the rail system and/or that the support profile is firmly anchored relative to the base surface. In particular, the measuring track groove and the rail groove are arranged substantially parallel to one another. In particular, the measuring track can be adhesively connected to the support profile.

One embodiment provides that the medical imaging system also has a data processing unit and that the data processing unit is configured to calculate a drive signal on the basis of the position information item, wherein the carriage has a travel drive, wherein the travel drive is configured to generate the drive force for the translation movement of the computed tomography gantry dependent upon the drive signal.

In particular, it can be provided that the wheel direct drives of the wheels of the set of wheels together form the travel drive.

A medical system is also disclosed herein, having a support construction, a carriage and a rail system and a support profile,
wherein the support construction is supported via the carriage and the rail system, able to be moved such that a translation movement of the support construction relative to a base surface can be carried out along the rail system,
wherein the support profile is received in a form-fitting manner into a depression that is formed relative to the base surface,
wherein the support profile has a rail groove for form-fitting receiving of a rail of the rail system,
wherein the rail of the rail system is received in a form-fitting manner into the rail groove such that the rail of the rail system does not protrude above the base surface in relation to a vertical direction.

A medical system is also disclosed herein, having a support construction, a carriage, a rail system and a support profile,
wherein the support construction is supported via the carriage and the rail system, able to be moved such that a translation movement of the support construction relative to a base surface can be carried out along the rail system,
wherein the support profile is received in a form-fitting manner into a depression that is formed relative to the base surface,
wherein the support profile has a rail groove for form-fitting receiving of a rail of the rail system,
wherein, in the region of the rail groove, the support profile has a profile-side connecting element,
wherein the rail of the rail system has a rail-side connecting element that is configured corresponding to the profile-side connecting element,
wherein the rail of the rail system is secured by way of a form-fitting of the profile-side connecting element and of the rail-side connecting element against a rotation about a longitudinal axis of the rail of the rail system relative to the support profile.

Optionally, it is provided that the carriage and the rail system are configured to transmit a drive force for the translation movement of the support construction in a force-locked manner from the carriage to the rail system.

The medical system with the support construction can be configured, for example, analogously to one of the aspects that are described for the medical imaging system with the computed tomography gantry. The medical system can be, for example, an X-ray imaging system, in particular, with a C-arm as the support construction, a magnetic resonance imaging system, in particular, with a support construction holding the body coil, a radiation therapy device, in particular, with a support construction holding the radiation source, or a patient positioning apparatus, in particular, with a patient bed as the support construction.

In the context of the invention, features which are described in relation to different embodiments of the invention and/or different claim categories (method, use, apparatus, system, arrangement, etc.) can be combined to form further embodiments of the invention. For example, a claim which relates to an apparatus, can also be further developed with features which are described or claimed in relation to a method and vice versa. Functional features of a method can be carried out via correspondingly configured physical components. The use of the indefinite article “a” or “an” does not preclude that the relevant feature can also be present plurally.

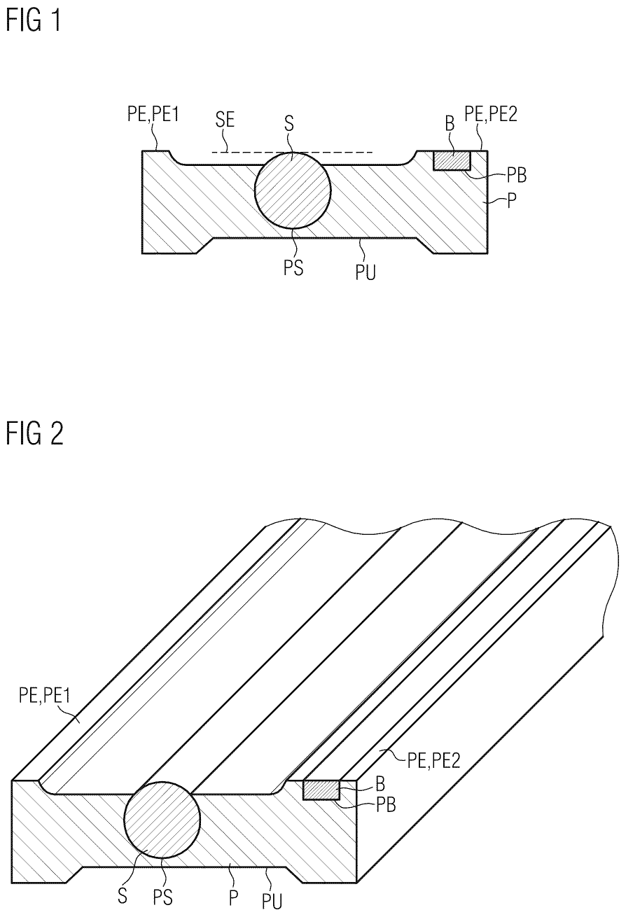

FIG. 1 shows the support profile P, the rail S and the measuring track B in a first view, wherein the support profile P has a measuring track groove PB for form-fitting receiving of the measuring track B and the measuring track B is received in a form-fitting manner into the measuring track groove PB, wherein the support profile P has a rail groove PS for form-fitting receiving of a rail S of the rail system L and the rail S of the rail system L is received into the rail groove PS in a form-fitting manner. The support profile P has the anchoring structure PU for form-fitting anchoring in a corresponding cut-out of the base surface U. FIG. 2 shows the support profile P, the rail S and the measuring track B in a second view.

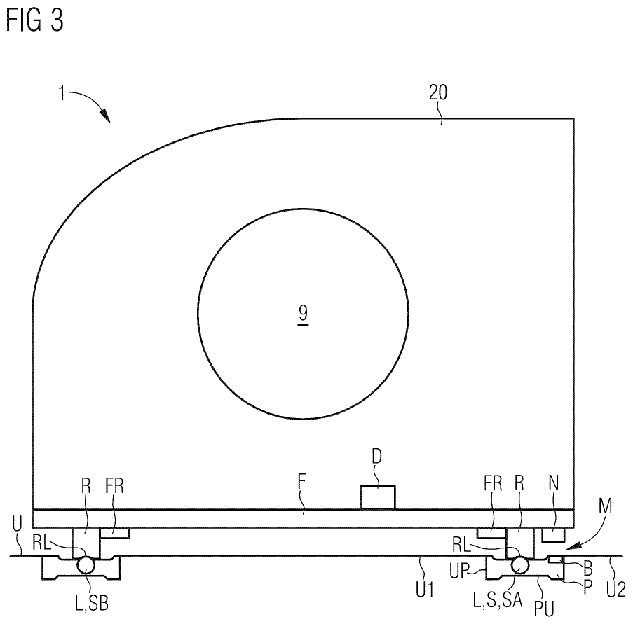

FIG. 3 shows the medical imaging system 1, having the computed tomography gantry 20, the carriage F and the rail system L, wherein the computed tomography gantry 20 is supported via the carriage F and the rail system L, able to be moved such that a translation movement of the computed tomography gantry 20 can be carried out along the rail system L, wherein the carriage F and the rail system L are configured to transmit a drive force for the translation movement of the computed tomography gantry 20 in a force-locked manner from the carriage F to the rail system L.

Provided between the carriage F and the rail system L is a set of wheel-rail rolling contacts RL, wherein the carriage F and the rail system L are configured to transmit the drive force for the translation movement of the computed tomography gantry 20 via the set of wheel-rail rolling contacts RL in a force-locked manner from the carriage F to the rail system L. The set of wheel-rail rolling contacts RL receives the total weight force of the carriage F and the computed tomography gantry 20, wherein each wheel-rail rolling contact that is included in the set of wheel-rail rolling contacts RL and receives at least a part of the total weight force of the carriage F and of the computed tomography gantry 20 transfers at least a part of the drive force for the translation movement of the computed tomography gantry 20 in a force-locked manner. The rail system L has a set of rails wherein the carriage F has a set of wheels R, wherein the set of wheels R is arranged rolling on the set of rails. For each wheel of the set of wheels R, the carriage F has a wheel direct drive that cooperates with this wheel and contributes proportionally to the drive force for the translation movement of the computed tomography gantry 20.

The medical imaging system 1 also has the position measurement system M, wherein the position measurement system M is configured for generating S2 a position information item, wherein the position information item relates to a position of the computed tomography gantry 20 along the rail system L. The position measurement system M is configured to generate S2 the position information item on the basis of a contactless measurement of the position of the computed tomography gantry 20 along the rail system L. The position measurement system M has the measuring track B and the position sensor N, wherein the measuring track B is static relative to the rail system L and extends along the rail system L, wherein the position sensor N is connected to the carriage F in such a way that it follows the translation movement of the computed tomography gantry 20 and, during the translation movement of the computed tomography gantry 20, cooperates with the measuring track B. For example, in the region of the other rail of the rail system L (in the left part of FIG. 3), a corresponding position measurement system can also be provided, in particular, with a measuring track and a position sensor.

The medical imaging system 1 also has a data processing unit D, wherein the data processing unit D is configured to calculate a drive signal on the basis of the position information item, wherein the carriage F has a travel drive FR, wherein the travel drive FR is configured to generate the drive force for the translation movement of the computed tomography gantry 20 dependent upon the drive signal.

The computed tomography gantry 20 has the opening 9. By way of the translation movement of the computed tomography gantry 20, an examination table can be introduced into the opening 9, in particular, together with an examination object supported on the examination table.

The example shown relates to a medical imaging system 1 having a computed tomography gantry 20, a carriage F, a rail system L and a support profile P,
wherein the computed tomography gantry 20 is mounted able to be moved via the carriage F and the rail system L such that a translation movement of the computed tomography gantry 20 relative to a base surface U can be carried out along the rail system L,
wherein the support profile P is received in a form-fitting manner into a depression UP that is formed relative to the base surface U,
wherein the support profile P has a rail groove PS for form-fitting receiving of a rail S of the rail system L,
wherein the rail S of the rail system L is received in a form-fitting manner into the rail groove PS such that the rail S of the rail system L does not protrude above the base surface U in relation to a vertical direction.

The example shown provides that no tangent plane SE to the rail S of the rail system L that is parallel to the base surface U extends above the base surface U.

The example shown provides that the base surface U has a first base surface region U1 and a second base surface region U2,
wherein the first base surface region U1 and the second base surface region U2 are coplanar with one another,
wherein the depression UP is situated between the first base surface region U1 and the second base surface region U2,
wherein the rail S of the rail system L is received in a form-fitting manner into the rail groove PS such that the rail S of the rail system L does not protrude above the first base surface region U1 and that the rail S of the rail system L does not protrude above the second base surface region U2.

The example shown provides that the support profile P has a connecting area PE,
wherein the support profile P is received in a form-fitting manner into the depression UP such that the connecting area PE is joined steplessly, in particular, in coplanar manner, to the base surface U, in particular, as a continuation of the base surface U.

The example shown provides that the connecting area PE has a first connecting area region PE1 and a second connecting area region PE2,
wherein the rail S of the rail system L is situated between the first connecting area region PE1 and the second connecting area region PE2,
wherein the support profile P is received in a form-fitting manner into the depression UP such that the first connecting area region PE1 is joined steplessly, in particular, in coplanar manner, to the first base surface region U1, in particular, as a continuation of the first base surface region U1, and that the second connecting area region PE2 is joined steplessly, in particular, in coplanar manner, to the second base surface region U2, in particular, as a continuation of the second base surface region U2.

The example shown provides that the base surface U extends substantially horizontally,
wherein the rail S of the rail system L is received in a form-fitting manner into the rail groove PS such that the rail S of the rail system L does not protrude above the base surface U in relation to the vertical direction.

The example shown provides that the rail S of the rail system L is a first rail SA of the rail system L and is arranged parallel to a second rail SB of the rail system L,
wherein the base surface U is substantially parallel to a rail plane, wherein the rail plane extends through the first rail SA of the rail system L and through the second rail SB of the rail system L.

The example shown provides that, in a transverse plane that is perpendicular to a longitudinal direction of the rail S of the rail system L, the rail S of the rail system L has a round profile, in particular, a circular profile.

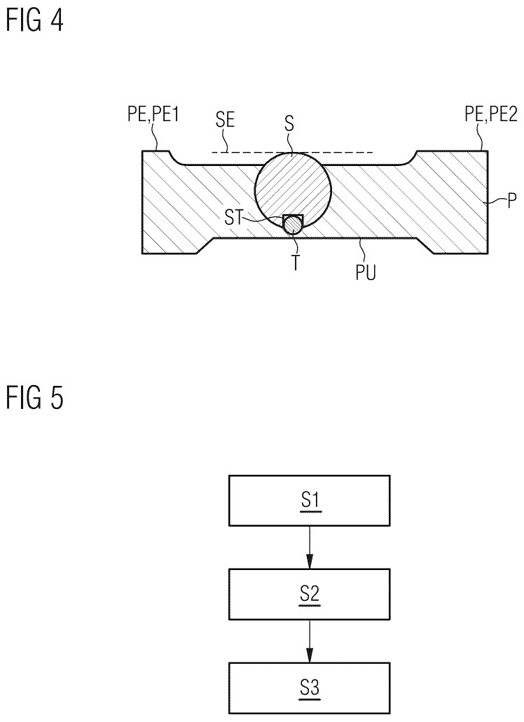

The example shown in FIG. 4 provides that the support profile P has a profile-side connecting element T in the region of the rail groove PS,
wherein the rail S of the rail system L has a rail-side connecting element ST that is configured corresponding to the profile-side connecting element T,
wherein the rail S of the rail system L is secured by way of a form-fitting of the profile-side connecting element T and of the rail-side connecting element ST against a rotation about a longitudinal axis of the rail S of the rail system L relative to the support profile P.

The example shown provides that the profile-side connecting element T protrudes in a rod-like manner toward the rail S of the rail system L in a direction that is perpendicular to the longitudinal axis of the rail S of the rail system L,
wherein the rail-side connecting element ST has a cut-out for receiving the profile-side connecting element T.

The example shown provides that the cut-out is a bore, in particular, in the form of a circular hole or slot and/or that the cut-out extends longitudinally parallel to the longitudinal axis of the rail S of the rail system L.

FIG. 5 shows a flow diagram of a method for moving a computed tomography gantry 20, wherein the computed tomography gantry 20 is mounted able to be moved via a carriage F and a rail system L such that a translation movement of the computed tomography gantry 20 along the rail system L can be carried out, the method comprising:
an execution S1 of the translation movement of the computed tomography gantry 20 along the rail system L, wherein a drive force for the translation movement of the computed tomography gantry 20 in a force-locked manner is transmitted in a force-locked manner from the carriage F to the rail system L,
a generation S2 of a position information item via a position measurement system M during the execution S1 of the translation movement of the computed tomography gantry 20 along the rail system L, wherein the position information item relates to a position of the computed tomography gantry 20 along the rail system L,
a provision S3 of the position information item.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

When a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc).

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A medical imaging system comprising:
a computed tomography gantry;
a carriage;
a rail system; and
a support profile,
wherein the computed tomography gantry is moveable via the carriage and the rail system such that a translation movement of the computed tomography gantry relative to a base surface occurs along the rail system,
the support profile is form-fit into a depression that is relative to the base surface,
the support profile has a rail groove to form-fit receive a rail of the rail system, and
the rail of the rail system is form-fit into the rail groove such that the rail of the rail system does not protrude above the base surface in relation to a vertical direction.

2. The medical imaging system of claim 1, wherein no tangent plane to the rail of the rail system that is parallel to the base surface extends above the base surface.

3. The medical imaging system of claim 1, wherein
the base surface has a first base surface region and a second base surface region,
the first base surface region and the second base surface region are coplanar,
the depression is between the first base surface region and the second base surface region, and
the rail of the rail system is form-fit into the rail groove such that the rail of the rail system does not protrude above the first base surface region and that the rail of the rail system does not protrude above the second base surface region.

4. The medical imaging system of claim 1, wherein
the support profile has a connecting area, and
the support profile is form-fit into the depression such that the connecting area is joined steplessly to the base surface.

5. The medical imaging system of claim 3, wherein
the support profile has a connecting area and the connecting area has a first connecting area region and a second connecting area region,
the rail of the rail system is between the first connecting area region and the second connecting area region, and
the support profile is form-fit into the depression such that the first connecting area region is joined steplessly to the first base surface region and that the second connecting area region is joined steplessly to the second base surface region.

6. The medical imaging system of claim 1, wherein
the base surface extends horizontally, and
the rail of the rail system is form-fit into the rail groove such that the rail of the rail system does not protrude above the base surface in relation to the vertical direction.

7. The medical imaging system of claim 1, wherein
the rail of the rail system is a first rail of the rail system and is parallel to a second rail of the rail system,
the base surface is substantially parallel to a rail plane, and
the rail plane extends through the first rail of the rail system and through the second rail of the rail system.

8. The medical imaging system of claim 1, wherein, in a transverse plane that is perpendicular to a longitudinal direction of the rail of the rail system, the rail of the rail system has a round profile.

9. The medical imaging system of claim 1, wherein, in a transverse plane that is perpendicular to a longitudinal direction of the rail of the rail system, the rail of the rail system has a rounded rectangular profile.

10. The medical imaging system of claim 1, wherein
the support profile has a profile-side connecting element in a region of the rail groove,
the rail of the rail system has a rail-side connecting element corresponding to the profile-side connecting element, and
the rail of the rail system is secured by a form-fitting of the profile-side connecting element and of the rail-side connecting element against a rotation about a longitudinal axis of the rail of the rail system relative to the support profile.

11. A medical imaging system comprising:
a computed tomography gantry;
a carriage;
a rail system; and
a support profile,
wherein the computed tomography gantry is moveable via the carriage and the rail system such that a translation movement of the computed tomography gantry relative to a base surface can be carried out along the rail system,
the support profile is form-fit into a depression that is relative to the base surface,
the support profile has a rail groove to form-fit receive a rail of the rail system,
the support profile has a profile-side connecting element in a region of the rail groove,
the rail of the rail system has a rail-side connecting element corresponding to the profile-side connecting element, and
the rail of the rail system is secured by a form-fitting of the profile-side connecting element and of the rail-side connecting element against a rotation about a longitudinal axis of the rail of the rail system relative to the support profile.

12. The medical imaging system of claim 11, wherein
the profile-side connecting element protrudes in a rod-like manner toward the rail of the rail system in a direction that is perpendicular to the longitudinal axis of the rail of the rail system, and
the rail-side connecting element has a cut-out to receive the profile-side connecting element.

13. The medical imaging system of claim 11, wherein
the rail-side connecting element protrudes in a rod-like manner away from the rail of the rail system in a direction that is perpendicular to the longitudinal axis of the rail of the rail system, and
the profile-side connecting element has a cut-out to receive the rail-side connecting element.

14. The medical imaging system of claim 12, wherein the cut-out is a bore.

15. The medical imaging system of claim 13, wherein the cut-out is a bore.

16. The medical imaging system of claim 12, wherein the cut-out is at least one of a slot or extending longitudinally parallel to the longitudinal axis of the rail of the rail system.

17. The medical imaging system of claim 13, wherein the cut-out is at least one of a slot or extending longitudinally parallel to the longitudinal axis of the rail of the rail system.

18. The medical imaging system of claim 2, wherein
the base surface has a first base surface region and a second base surface region,
the first base surface region and the second base surface region are coplanar with one another,
the depression is between the first base surface region and the second base surface region, and
the rail of the rail system is form-fit into the rail groove such that the rail of the rail system does not protrude above the first base surface region and that the rail of the rail system does not protrude above the second base surface region.

19. The medical imaging system of claim 18, wherein
the support profile has a connecting area, and
the support profile is form-fit into the depression such that the connecting area is joined steplessly to the base surface.

20. The medical imaging system of claim 19, wherein
the connecting area has a first connecting area region and a second connecting area region,
the rail of the rail system is situated between the first connecting area region and the second connecting area region, and
the support profile is form-fit into the depression such that the first connecting area region is joined steplessly to the first base surface region and that the second connecting area region is joined steplessly to the second base surface region.
